(19) United States
(12) Patent Application Publication
(10) Pub. No.: US 2014/0213870 A1
(43) Pub. Date: Jul. 31, 2014

(54) Non-Invasive Blood glucose Sensor

(71) Applicant: LUNGWHA UNIVERSITY OF SCIENCE AND TECHNOLOGY, Taoyuan (TW)

(72) Inventors: Chen Hsu, Taoyuan (TW); Tzu-Hsiang Ko, Taoyuan (TW); Ru-Jen Lin, Taoyuan (TW); An-Doo Yang, Taoyuan (TW); Ya-Dong Pan, Taoyuan (TW); Siang-Yu Lin, Taoyuan (TW)

(73) Assignee: Lungwha University of Science and Technology, Taoyuan (TW)

(21) Appl. No.: 13/754,880

(22) Filed: Jan. 30, 2013

(51) Int. Cl. A61B 5/145; A61B 5/00

(52) U.S. Cl. CPC A61B 5/14532; A61B 5/0004; A61B 5/6801
USPC 600/365

(57) ABSTRACT

The present invention relates to a non-invasive blood glucose sensor, comprising: a substrate, a first metal layer, a second metal layer, and a blood glucose sensing unit, wherein the first metal layer is formed on the one surface of the substrate and has a microstrip antenna in the internal thereof, the second metal layer is formed on the other surface of the substrate, and the blood glucose sensing unit is electrically connected to the first metal layer and the second metal layer. In the present invention, the non-invasive blood glucose sensor can be used to measure a numerical value of the blood glucose in a human body by way of disposing the non-invasive blood glucose sensor near the human body, without using any body-invading ways, for example, the acupuncture treatment; therefore the inconveniences and incorrect measurements resulting from the body-invading ways can be avoided.

### BACKGROUND OF THE INVENTION

1. Technical Field

The present invention relates to a blood glucose sensor, and more particularly to a non-invasive blood glucose sensor able to be used for measuring a numerical value of the blood glucose in a human body by way of disposing the non-invasive blood glucose sensor near the human body.

2. Description of Related Art

With the development of science and technology, the diet and exercise habits of people is much different from the diet and exercise habits in the past, so the chronic disease become inevitable to everyone in this civilization society. The diabetes is one of many common chronic diseases which are easy to cause serious complications, such as retinopathy, nephropathy, high blood pressure, etc. Although there is still no cure method of diabetes, the blood glucose concentration could be also effectively controlled by diet, exercise and drug. Therefore, it's important to measure the blood glucose concentration regularly to control the blood glucose concentration.

The method of measuring blood glucose concentration in the present is invading the body by a needle, and sampling the blood, then analyzing the blood to get the blood glucose concentration. The method of body-invading by acupuncture treatment would cause the fear feeling to the patients, thus the accuracy of measurement would be effected (such as the acupuncture treatment does not insert the skin correctly, then the patient must further extrude the blood).

Therefore, the method of non-invading blood glucose measurement is a very important way for patients to avoid the fear feeling of measurement, then the diabetes would be controlled effectively for avoiding the complications or fatality of diabetes.

Accordingly, in view of the conventional method of invading the body still has shortcomings and drawback, the inventor of the present application has made great efforts to make inventive research thereon and eventually provided a non-invasive blood glucose sensor.

### BRIEF SUMMARY OF THE INVENTION

The primary objective of the present invention is to provide a non-invasive blood glucose sensor for being used to measure a numerical value of the blood glucose in a human body by way of disposing the non-invasive blood glucose sensor near the human body, without using any body-invading ways, for example, the acupuncture treatment; therefore the inconveniences and incorrect measurements resulting from the body-invading ways can be avoided.

Thus, for achieving the objective of the present invention, the inventors of the present invention propose a non-invasive blood glucose sensor, comprising:
a substrate;
a first metal layer, formed on the one surface of the substrate and having a microstrip antenna in the internal thereof;
a second metal layer, formed on the other surface of the substrate; and
a blood glucose sensing unit, electrically connected to the first metal layer and the second metal layer and capable of providing an RF signal; wherein when a user disposes the non-invasive blood glucose sensor near a human body, the blood glucose sensing unit would output the RF signal to the first metal layer, therefore a resonance is produced by the first metal layer with the RF signal and a blood glucose in the human body, and then the numerical value of the blood glucose is calculated and display by the blood glucose sensing unit;
wherein, opposite to the substrate, an overlapping area and a non-overlapping are provided between the first metal layer and the second metal layer for improving the bandwidth of the microstrip antenna and the sensing sensitivity of the blood glucose sensing unit.

### BRIEF DESCRIPTION OF THE SEVERAL VIEWS OF THE DRAWINGS

The invention as well as a preferred mode of use and advantages thereof will be best understood by referring to the following detailed description of an illustrative embodiment in conjunction with the accompanying drawings, wherein:

FIG. 1 is a block diagram of the a non-invasive blood glucose sensor according to the present invention;

FIG. 2 is a top view of the non-invasive blood glucose sensor according to the present invention;

FIG. 3 is an exploded view of the non-invasive blood glucose sensor according to the present invention;

FIG. 4 is a first measurement data curve plot measured by using the non-invasive blood glucose sensor;

FIG. 5 is a second measurement data curve plot measured by using the non-invasive blood glucose sensor; and

FIG. 6 is a third measurement data curve plot measured by using the non-invasive blood glucose sensor.

### DETAILED DESCRIPTION OF THE INVENTION

To more clearly describe a non-invasive blood glucose sensor according to the present invention, embodiments of the present invention will be described in detail with reference to the attached drawings hereinafter.

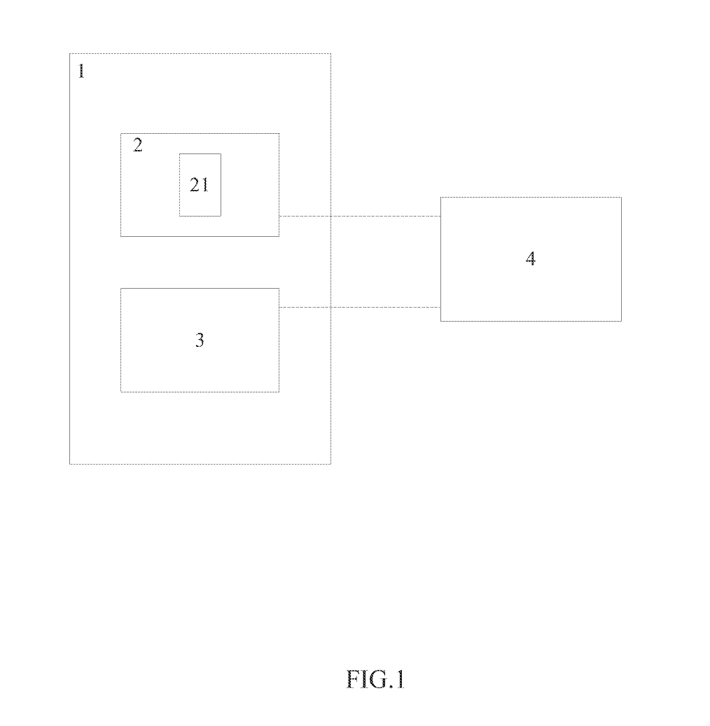

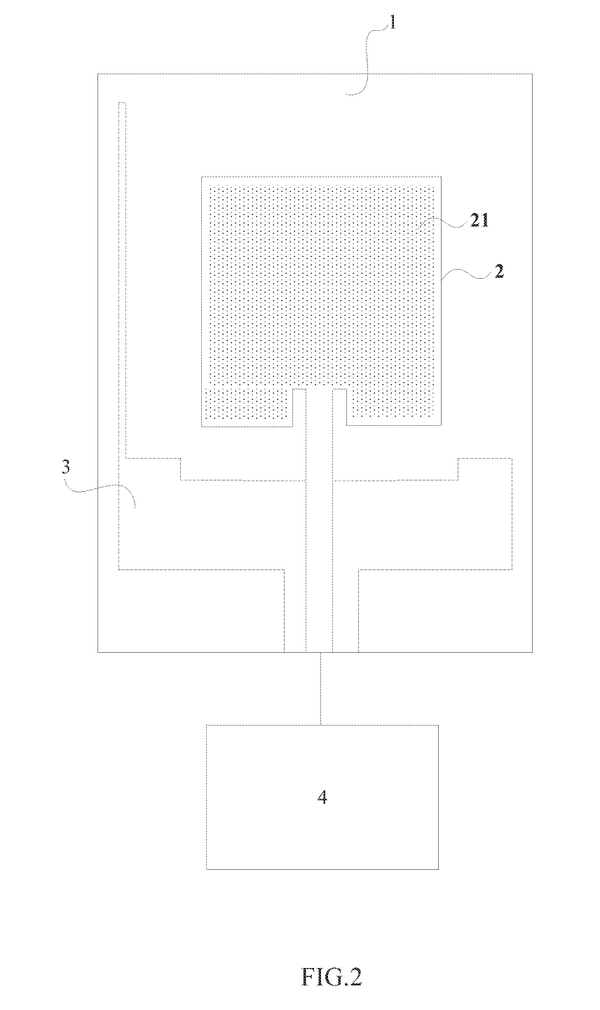

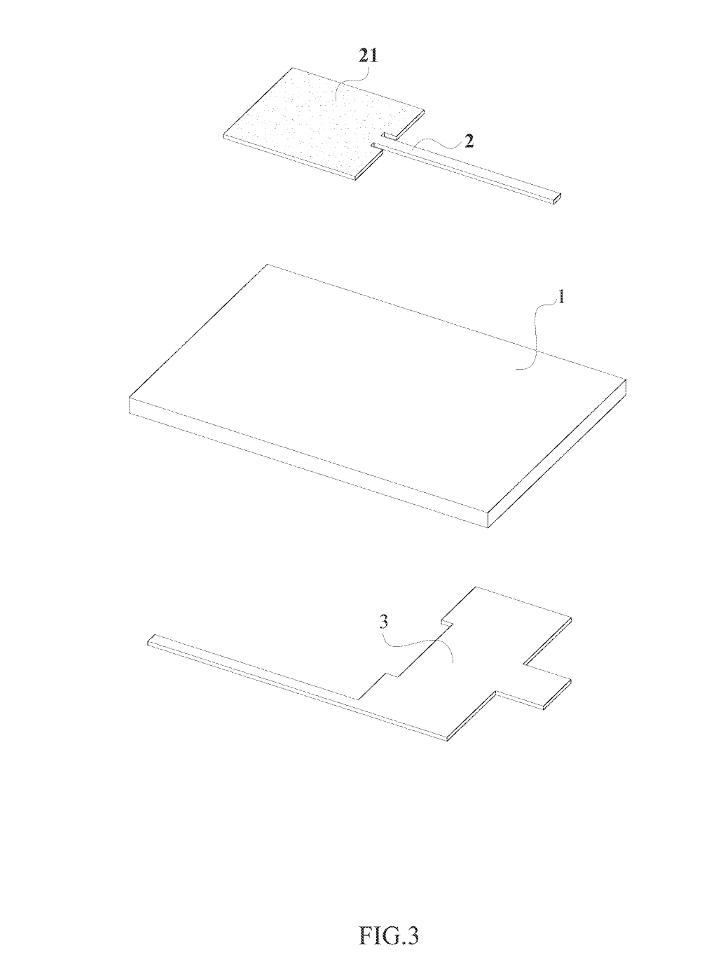

First of all, the framework and structure of the non-invasive blood glucose sensor will be described. With reference to FIG. 1, FIG. 2 and FIG. 3, there are shown a block diagram, a top view, and an exploded view of the non-invasive blood glucose sensor according to the present invention. As shown in FIGs, the non-invasive blood glucose sensor consists of a substrate 1, a first metal layer 2, a second metal layer 3, a conductive coating layer 21, and a blood glucose sensing unit 4, wherein the substrate 1 may be a polymer substrate or a ceramic substrate.

In this non-invasive blood glucose sensor, the first metal layer 2 is formed on the one surface of the substrate 1 and has a microstrip antenna in the internal thereof, moreover, there are nano metal particles mixed in the conductive coating layer 21 selectively, which are used for increasing the sensing sensitivity of the non-invasive blood glucose sensor.

The second metal layer 3 is formed on the other surface of the substrate 1; and particularly, opposite to the substrate 1, an overlapping area and a non-overlapping are provided between the first metal layer 2 and the second metal layer 3 for improving the bandwidth of the microstrip antenna and the sensing sensitivity of the blood glucose sensing unit 1; in addition, the overlapping area and non-overlapping are produced by modulating the shape of the first metal layer and the second metal layer. As shown in FIG. 2, the second metal layer 3 is exemplary an L-shaped metal layer consisting a certain area, and the certain area does not overlap with the first metal layer 2; moreover, opposite to the substrate 1, the otherwise area the first metal layer 2 is disposed above the concave part of the L-shaped second metal layer 3 for improving the bandwidth of the microstrip antenna, and making the resonance frequency be ranged between 1 GHZ and 8 GHZ.

The blood glucose sensing unit 4 is electrically connected to the first metal layer 2 and the second metal layer 3, and capable of providing an RF signal. In the present invention, when a user disposes the non-invasive blood glucose sensor near a human body, the blood glucose sensing unit 4 would output the RF signal to the first metal layer 2, therefore a resonance with the resonance frequency ranged between 1 GHZ and 8 GHZ is produced by the first metal layer 2 with the RF signal and a blood glucose in the human body, and then the numerical value of the blood glucose is calculated and display by the blood glucose sensing unit 4.

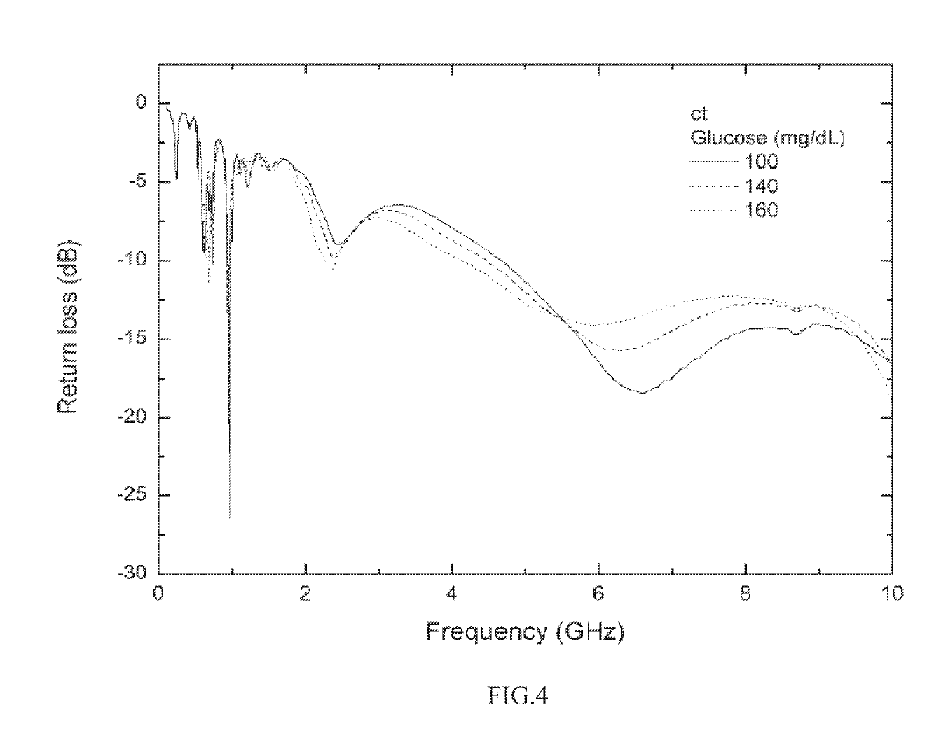

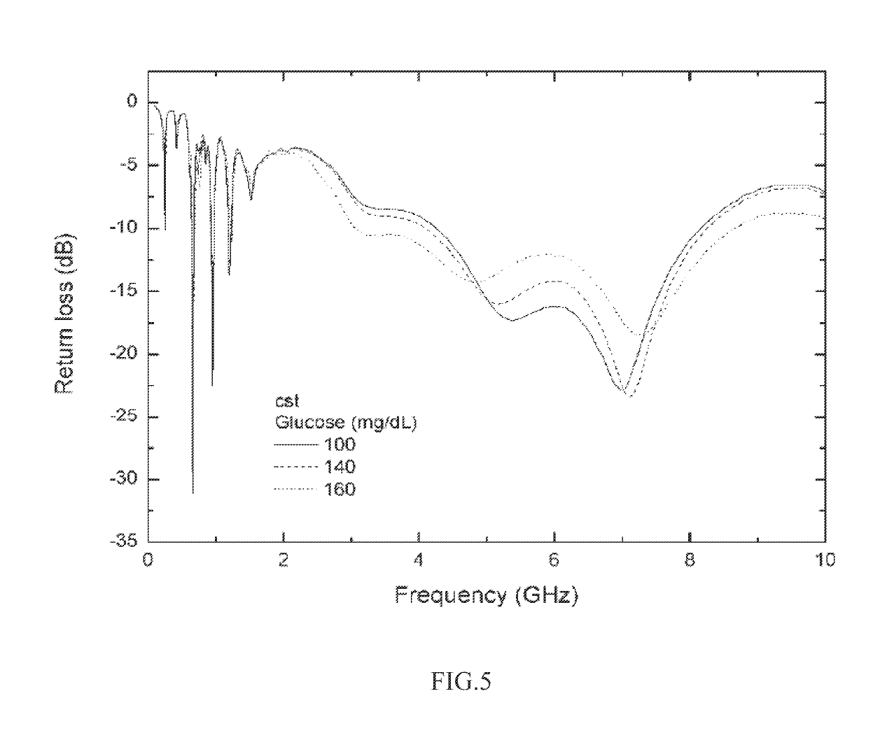

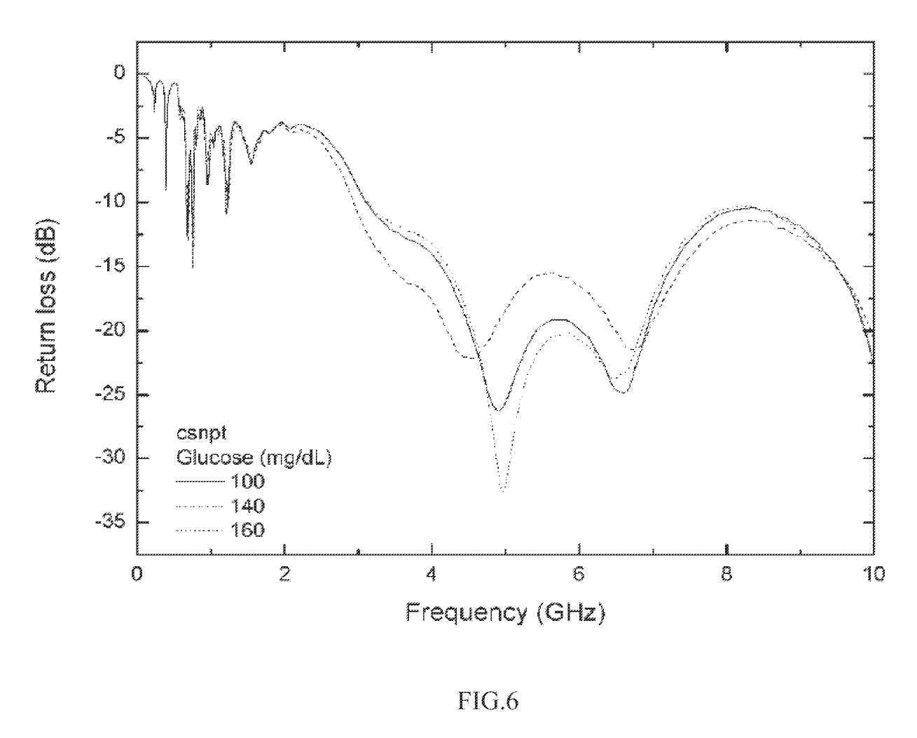

Therefore, through above descriptions, the framework and structure of the non-invasive blood glucose sensor have been introduced completely and clearly. Next, the precision of the blood glucose value measured by non-invasive blood glucose sensor will be proven through various experiment data. With reference to FIG. 4, FIG. 5 and FIG. 6, there are shown a first measurement data curve plot, a second measurement data curve plot and a third measurement data curve plot measured by using the non-invasive blood glucose sensor of the present invention. In which, the first measurement data is measured by using the non-invasive blood glucose sensor, and the non-invasive blood glucose sensor (called the first embodiment non-invasive blood glucose sensor hereinafter) consists of the substrate 1, the first metal layer 2 and the second metal layer 3, but does not include the conductive coating layer 21 on the first metal layer 2. From FIG. 4, three return loss curves are measured after the non-invasive blood glucose sensor produces the various resonances according to three different blood glucose concentrations of 100 mg/dL, 140 mg/dL and 160 mg/dL; and as shown in FIG. 4, the amplitude of the return loss is about −29 db in the frequency between 1 GHZ and 8 GHZ, and such amplitude of the return loss is large enough to be easily identify.

Moreover, the second measurement data is measured by using the non-invasive blood glucose sensor, and the non-invasive blood glucose sensor 1 (called the second embodiment non-invasive blood glucose sensor hereinafter) includes the substrate, the first metal layer 2 and the second metal layer 3, wherein the conductive coating layer 21 is coated on the first metal layer 2. From FIG. 5, three return loss curves are measured after the non-invasive blood glucose sensor produces the various resonances according to three different blood glucose concentrations of 100 mg/dL, 140 mg/dL and 160 mg/dL; and as shown in FIG. 5, the amplitude of the return loss is about −31 db in the frequency between 1 GHZ and 8 GHZ, and such amplitude of the return loss can also be easily identify.

Furthermore, the second measurement data is measured by using the non-invasive blood glucose sensor, and the non-invasive blood glucose sensor (called the third embodiment non-invasive blood glucose sensor hereinafter) has of the substrate 1, the first metal layer 2 and the second metal layer 3; in which, the conductive coating layer 21 is coated on the first metal layer 2, and some nano metal particles are mixed in the conductive coating layer 21. From FIG. 6, three return loss curves are measured after the non-invasive blood glucose sensor produces the various resonances according to three different blood glucose concentrations of 100 mg/dL, 140 mg/dL and 160 mg/dL; and as shown in FIG. 5, the amplitude of the return loss is about −32 db in the frequency between 1 GHZ and 8 GHZ, and such amplitude of the return loss can also be easily identify.

Thus, through the first, second and third experiment data, it can know that all the return loss amplitudes of FIG. 4, FIG. 5 and FIG. 6 are large enough to be easily identify; and accordingly, the greater the return loss amplitude shows, the better the sensing sensitivity that the non-invasive blood glucose sensor performs. Moreover, the sensing sensitivity of the second embodiment non-invasive blood glucose sensor is greater than the sensing sensitivity of the first embodiment non-invasive blood glucose sensor, and the sensing sensitivity of the third embodiment non-invasive blood glucose sensor is greater than the sensing sensitivity of the second embodiment non-invasive blood glucose sensor.

So that, according to above descriptions, the present invention has been completely and clearly disclosed; and in summary, the main advantage of the present invention is that a non-invasive blood glucose sensor 1 is provided and used for measuring a numerical value of the blood glucose in a human body by way of disposing the non-invasive blood glucose sensor near the human body, without using any body-invading ways, for example, the acupuncture treatment; therefore the inconveniences and incorrect measurements resulting from the body-invading ways can be avoided.

The above description is made on embodiments of the present invention. However, the embodiments are not intended to limit scope of the present invention, and all equivalent implementations or alterations within the spirit of the present invention still fall within the scope of the present invention.

We claim:

1. A non-invasive blood glucose sensor, comprising:
a substrate;
a first metal layer, being formed on the one surface of the substrate and having a microstrip antenna in the internal thereof;
a second metal layer, being formed on the other surface of the substrate; and
a blood glucose sensing unit, being electrically connected to the first metal layer and the second metal layer and capable of providing an RF signal; wherein when a user disposes the non-invasive blood glucose sensor near a human body, the blood glucose sensing unit would output the RF signal to the first metal layer, therefore a resonance is produced by the first metal layer with the RF signal and a blood glucose in the human body, and then the numerical value of the blood glucose is calculated and display by the blood glucose sensing unit;
wherein, opposite to the substrate, an overlapping area and a non-overlapping being provided between the first metal layer and the second metal layer for improving the bandwidth of the microstrip antenna and the sensing sensitivity of the blood glucose sensing unit.

2. The non-invasive blood glucose sensor of claim 1, wherein the substrate is selected from the group consisting of: polymer substrate and ceramic substrate.

3. The non-invasive blood glucose sensor of claim 1, wherein a conductive coating layer is further coated on the first metal layer for increasing the sensitivity of the non-invasive blood glucose sensor, and the conductive coating layer having a specific conductive coating area.

4. The non-invasive blood glucose sensor of claim 1, wherein the first metal layer and the second metal layer respectively have a specific shape for making the resonance frequency be ranged between 1 GHZ and 8 GHZ.

5. The non-invasive blood glucose sensor of claim 1, wherein the overlapping area and non-overlapping are produced by modulating the shape of the first metal layer and the second metal layer.

6. The non-invasive blood glucose sensor of claim 1, wherein the frequency of RF is ranged between 1 GHz and 8 GHz.

7. The non-invasive blood glucose sensor of claim 3, wherein the specific conductive coating area is larger than the area of the second metal layer.

8. The non-invasive blood glucose sensor of claim 3, wherein the specific conductive coating area is smaller than the area of the second metal layer.

9. A non-invasive blood glucose sensor, comprising:
a substrate;
a first metal layer, being formed on the one surface of the substrate and having a microstrip antenna in the internal thereof;
a second metal layer, being formed on the other surface of the substrate;
a conductive coating layer, being coated on the first metal layer for increasing the sensitivity of the non-invasive blood glucose sensor, and having a specific conductive coating area;
a blood glucose sensing unit, being electrically connected to the first metal layer and the second metal layer and capable of providing an RF signal; wherein when a user disposes the non-invasive blood glucose sensor near a human body, the blood glucose sensing unit would output the RF signal to the first metal layer, therefore a resonance is produced by the first metal layer with the RF signal and a blood glucose in the human body, and then the numerical value of the blood glucose is calculated and display by the blood glucose sensing unit;
wherein, opposite to the substrate, an overlapping area and a non-overlapping being provided between the first metal layer and the second metal layer for improving the bandwidth of the microstrip antenna and the sensing sensitivity of the blood glucose sensing unit.

10. The non-invasive blood glucose sensor of claim 9, wherein specific nano metal particles are further mixed in the conductive coating layer.

11. The non-invasive blood glucose sensor of claim 9, wherein the substrate is selected from the group consisting of: polymer substrate and ceramic substrate.

12. The non-invasive blood glucose sensor of claim 9, wherein the first metal layer and the second metal layer respectively have a specific shape for making the resonance frequency be ranged between 1 GHZ and 8 GHZ.

13. The non-invasive blood glucose sensor of claim 9, wherein the overlapping area and non-overlapping are produced by modulating the shape of the first metal layer and the second metal layer.

14. The non-invasive blood glucose sensor of claim 9, wherein the frequency of RF is ranged between 1 GHz and 8 GHz.
